(19) United States
(12) Patent Application Publication
(10) Pub. No.: US 2026/0232298 A1
(43) Pub. Date: Aug. 13, 2026

(54) SYSTEM AND METHOD FOR PREDICTING HIGH OVARIAN RESPONSE OF SUBJECT

(71) Applicants: PEKING UNI. THIRD HOSPITAL (THE THIRD CLINICAL MEDICAL SCHOOL OF PEKING UNI.), Beijing (CN); HANGZHOU QINGGUO MEDICAL TECHNOLOGY CO., LTD., Zhejiang (CN)

(72) Inventors: Huiyu XU, Beijing (CN); Rong LI, Beijing (CN); Jie QIAO, Beijing (CN); Guoshuang FENG, Beijing (CN); Yong HAN, Beijing (CN)

(73) Assignees: PEKING UNI. THIRD HOSPITAL (THE THIRD CLINICAL MEDICAL SCHOOL OF PEKING UNI.), Beijing (CN); HANGZHOU QINGGUO MEDICAL TECHNOLOGY CO., LTD., Zhejiang (CN)

(21) Appl. No.: 18/873,020

(22) PCT Filed: Aug. 17, 2022
(86) PCT No.: PCT/CN2022/113035

§ 371 Date: Aug. 8, 2025

(30) Foreign Application Priority Data
Feb. 18, 2022 (CN) 202210150332.2

(51) Int. Cl. A61B 10/00; G16H 50/30

(52) U.S. Cl. CPC A61B 10/0012; G16H 50/30

(57) ABSTRACT

The present application provides a system for predicting a high ovarian response of a subject, the system comprising: a data acquisition module which is used for acquiring the data of the subject including the basic anti-mullerian hormone (AMH) level and the serum inhibin B level; and a high ovarian response occurrence probability calculation module which is used for calculating said data acquired in the data acquisition module so as to calculate the high ovarian response occurrence probability of the subject. The data acquisition module further acquires the follicle-stimulating hormone (FSH) level, the androstenedione (AND) level, and the luteinizing hormone (LH) level of the subject; the subject is a subject who is to be subjected to standard ovarian stimulation treatment.

### TECHNICAL FIELD

The present application relates to the field of reproductive technology, and in particular to a system and method for predicting high ovarian response of a subject.

### BACKGROUND ART

In recent years, the incidence of infertility is about 8-10%, and shows an increasing trend year by year. Assisted reproductive technology (ART) has become the main means to solve infertility, in which IVF-ET is the core of assisted reproductive technology. During the controlled ovarian stimulation (COS) process, the number of retrieved oocytes is considered to be a powerful predictor of successful pregnancy after IVF-ET (in vitro fertilization-embryo transfer), that is, obtaining a sufficient number of high-quality oocytes is the key to determining the success rate of IVF-ET, and ovarian hyperstimulation is the prerequisite for obtaining high-quality and sufficient oocytes. Therefore, evaluating ovarian reserve and responsiveness has become a hot topic. In clinical work, as long as the patient has antral follicles in the ovaries, controlled ovarian hyperstimulation can be performed, and the responsiveness of the follicles to ovulation-stimulating drugs plays a decisive role. Therefore, accurate prediction of ovarian responsiveness is critical.

Ovarian responsiveness refers to the ability of the ovaries to respond to exogenous gonadotropins during controlled ovarian hyperstimulation (COH), and can be divided into low ovarian response, normal response and high ovarian response. Currently, there is no specific method to predict ovarian responsiveness. It is generally believed that the quality of ovarian responsiveness mainly depends on the ovarian reserve. Therefore, assessment indicators for ovarian reserve are often used to predict responsiveness.

About one-third of women undergoing IVF will experience high ovarian response (development of a large number of oocytes). This high ovarian response may result in poor embryo quality, reduced chance of pregnancy, or cycle cancellation. Additionally, patients with an overresponsive ovary are at risk of developing ovarian hyperstimulation syndrome (OHSS), a potentially life-threatening iatrogenic condition. In order to maximize the safety and effectiveness of assisted reproductive technology, it is necessary to identify patients at risk of high ovarian response at the beginning of ovarian stimulation treatment and take effective measures to prevent such excessive response.

### SUMMARY

Ovarian responsiveness, which refers to the ovarian response to ovarian stimulation (OS), is the cornerstone of in vitro fertilization (IVF) cycles and an independent risk factor for treatment success.

Although high ovarian response has no internationally recognized definition, it can be broadly considered as a situation in which the ovaries show more follicular growth than expected, is manifested by the recruitment and development of a large number of follicles (the number of developing follicles (follicles with a diameter of more than 10 mm)>20, and/or the number of retrieved oocytes >15) and a rapid rise in estrogen. High ovarian response itself does not necessarily cause problems, but it is a high-risk factor for women to develop clinical features of OHSS during ovarian hyperstimulation.

When the ovarian response is high, patients are more likely to experience overstimulation, which can lead to poor embryo quality, reduced pregnancy probability or cancellation of the gestational cycle. If patients with ovarian high response are not correctly identified and given conventional ovarian stimulation treatment during the controlled ovarian hyperstimulation (COH), they may develop ovarian hyperstimulation syndrome (OHSS), which can be life-threatening in severe cases, limiting the application of IVF-ET (in vitro fertilization-embryo transfer) surgery. Therefore, it is necessary to find appropriate sensitive indicators for predicting high ovarian response in order to guide clinical individualized controlled ovarian hyperstimulation treatment plans in the reproductive field. Giving an appropriate amount of Gn starting dose and subsequent daily doses to obtain a moderate number of high-quality oocytes is a key link in achieving clinical pregnancy and preventing high ovarian response.

As mentioned above, determining whether a subject has high ovarian response is a very important task for clinicians. However, there is no suitable system in the world to help clinicians determine the probability of high ovarian response in subjects. In the past, clinicians usually made judgments according to their clinical experience, based on the woman's history of ovarian response, age, BMI, AFC, and serum FSH level on days 2-4 of menstruation. Therefore, the present application is intended to provide a system in which the high ovarian response of a subject can be accurately, conveniently and quickly predicted.

Particularly, the present application relates to the following contents:

1. A system for predicting high ovarian response of a subject, comprising,
a data collection module, which is used for acquiring data of basic anti-mullerian hormone (AMH) level and serum inhibin B level of the subject; and
a module for calculating a probability of high ovarian response, which is used for performing calculation on the data acquired in the data collection module, so as to calculate the probability of high ovarian response of the subject.

2. The system according to item 1, wherein
the data collection module further acquires follicle-stimulating hormone (FSH) level, androstenedione (AND) level, and luteinizing hormone (LH) level of the subject; and
the subject is a subject who will receive standard ovarian stimulation treatment.

3. The system according to item 1, wherein, in the module for calculating a probability of high ovarian response, formula I for calculating the probability of high ovarian response of the subject is pre-stored, wherein the formula I is obtained by fitting based on data of a basal anti-Mullerian hormone (basal AMH) level and a serum inhibin B level change (ΔinhibinB) of a patient receiving standard ovarian stimulation treatment in an existing database.

4. The system according to item 2, wherein,
in module for calculating a probability of high ovarian response, formula II for calculating the probability of high ovarian response of the subject is pre-stored, wherein the formula II is obtained by fitting based on data of basal anti-Mullerian hormone (basal AMH) level, change in serum inhibin B level (ΔinhibinB), basal follicle-stimulating hormone (basal FSH) level, basal androstenedione (basal AND) level and change therein (ΔAND), and basal luteinizing hormone (basal LH) level of a patient receiving the standard ovarian stimulation treatment in an existing database.

5. The system according to any one of items 1 to 4, wherein,
in the data collection module,
the data of the basal anti-Mullerian hormone (basal AMH) level acquired refers to a concentration of anti-Mullerian hormone in venous blood of the subject on the 2nd day of menstrual period after receiving the ovarian stimulation treatment,
the data of the change in serum inhibin B level (ΔinhibinB) acquired refers to a difference between inhibin B concentrations in the venous blood of the subject on the 6th day and the 2nd day of the menstrual period after receiving the ovarian stimulation treatment,
the data of the basal follicle-stimulating hormone (basal FSH) level acquired refers to a concentration of FSH in the venous blood of the subject on the 2nd day of the menstrual period after receiving the ovarian stimulation treatment,
the data of the basal androstenedione (basal AND) level acquired refers to a concentration of androstenedione in the venous blood of the subject on the 2nd day of the menstrual period after receiving the ovarian stimulation treatment, and the data of the change in the androstenedione level (ΔAND) refers to a difference between the concentrations of androstenedione in the venous blood of the subject on the 6th day and the 2nd day of the menstrual period after receiving the ovarian stimulation treatment, or
the data of the basal luteinizing hormone (basal LH) level acquired refers to a concentration of LH in the venous blood of the subject on the 2nd day of the menstrual period after receiving the ovarian stimulation treatment.

6. The system according to any one of items 1 to 5, wherein,
in the module for calculating a probability of high ovarian response, the pre-stored formula I for calculating the probability of high ovarian response of the subject, which is obtained by fitting based on the data of the basal anti-Mullerian hormone (basal AMH) level and the change in inhibin B level (ΔinhibinB) of the patient receiving the standard ovarian stimulation treatment in the existing database, is a calculation formula which is obtained by fitting the data of anti-Mullerian hormone (AMH) level and change in INHBB level (ΔinhibinB) of the patient receiving the standard ovarian stimulation treatment in the existing database using logistic regression,
the formula I can be used to calculate the probability of high ovarian response of the subject by using the data of the basal anti-Mullerian hormone (basal AMH) level and dynamic change in inhibin B level (ΔinhibinB) of the subject acquired by the data collection module.

7. The system according to any one of items 4 to 6, wherein,
in the module for calculating a probability of high ovarian response, the pre-stored formula II for calculating the probability of high ovarian response of the subject, which is obtained by fitting based on the data of the basal anti-Mullerian hormone (basal AMH) level, the change in inhibin B level (ΔinhibinB), the basal follicle-stimulating hormone (basal FSH) level, the basal androstenedione (basal AND) level and the change therein (ΔAND), and the basal luteinizing hormone (basal LH) level of the patient receiving the standard ovarian stimulation treatment in the existing database, is a calculation formula which is obtained by fitting the data of the basal anti-Mullerian hormone (basal AMH) level, the change in inhibin level (ΔinhibinB), the basal follicle-stimulating hormone (basalFSH) level, the basal androstenedione (basal AND) level and the change therein (ΔAND), and the basal luteinizing hormone (basal LH) level of the patient receiving the standard ovarian stimulation treatment in the existing database using logistic regression;
the formula II can be used to calculate the probability of high ovarian response of the subject by using the data of basal anti-Mullerian hormone (basal AMH) level, the change in inhibin B level (ΔinhibinB), the basal follicle-stimulating hormone (basal FSH) level, the basal androstenedione (basal AND) level and the change therein (ΔAND), and the basal luteinizing hormone (basal LH) level of the subject acquired by the data collection module.

8. The system according to item 6, wherein:
the formula I is:

$$P = 1 / ( 1 + {e}^{( - ( a + b * ln ( basal AMH ) + c * ln ( Δ inhibinB ) ) )} ) ,$$

wherein
p is a parameter calculated to characterize the probability of high ovarian response of the subject;
a is any value selected from a range of −18.49449 to −9.790517, preferably −14.1425;
b is any value selected from a range of 0.4227336 to 1.548814, preferably 0.9857738;
c is any value selected from a range of 0.9233156 to 2.2649428, preferably 1.5941292; and
e is a natural number.

9. The system according to item 7, wherein:
the formula II is:

$$P = 1 / ( 1 + {e}^{( - ( d + f * basal FSH + g * ln ( basal AMH ) + h * basal AND + i * ln ( Δ inhibinB ) + j * Δ AND + k * basal LH ) )} ) ,$$

wherein
p is a parameter calculated to characterize the probability of high ovarian response of the subject,
d is any value selected from a range of −15.39228 to −6.729412, preferably −11.06085;
f is any value selected from a range of −0.589993 to −0.09121, preferably −0.340602;
g is any value selected from a range of 0.1606959 to 1.369763, preferably 0.7652294;
h is any value selected from a range of −0.013212 to 0.1226672, preferably 0.0547278;
i is any value selected from a range of 0.65715 to 2.0135819, preferably 1.3353659;
j is any value selected from a range of −0.036413 to 0.1330693, preferably 0.0483282;
k is any value selected from a range of −0.051137 to 0.2291053, preferably 0.088984; and
e is a natural number.

10. The system according to any one of items 4 to 9, wherein,
the module for calculating a probability of high ovarian response also comprises a comparison submodule, in which probability values of high ovarian response of the subject calculated by formula I and formula II respectively are compared, and a larger probability value is taken as the probability of high ovarian response of the subject.

11. A method for predicting high ovarian response in a subject, comprising:
a data collection step, in which data of basal anti-Mullerian hormone (AMH) level and serum inhibin B level of the subject is acquired; and
a step for calculating a probability of high ovarian response, in which calculation is performed on the data acquired in the data collection step, so as to calculate the probability of high ovarian response of the subject.

12. The method according to item 11, wherein,
in the data collection step, follicle-stimulating hormone (FSH) level, androstenedione (AND) level, and luteinizing hormone (LH) level of the subject are further acquired;
the subject is a subject who will receive standard ovarian stimulation treatment.

13. The method according to item 11, wherein,
in the step for calculating a probability of high ovarian response, formula I for calculating the probability of high ovarian response of the subject is pre-stored for use, wherein the formula I is obtained by fitting based on data of basal anti-Mullerian hormone (basal AMH) level and change in serum inhibin B level (ΔinhibinB) of a patient receiving standard ovarian stimulation treatment in an existing database.

14. The method according to item 12, wherein,
in the step for calculating a probability of high ovarian response, formula II for calculating the probability of high ovarian response of the subject is pre-stored for use, wherein the formula II is obtained by fitting based on data of basal anti-Mullerian hormone (basal AMH) level, change in serum inhibin B level (ΔinhibinB), basal follicle-stimulating hormone (basal FSH) level, basal androstenedione (basal AND) level and change therein (ΔAND), and basal luteinizing hormone (basal LH) level of a patient receiving the standard ovarian stimulation treatment in an existing database.

15. The method according to any one of items 11 to 14, wherein,
in the data collection step,
the data of the basal anti-Mullerian hormone (basalAMH) level acquired refers to a concentration of anti-Mullerian hormone in venous blood of the subject on the 2nd day of menstrual period after receiving the ovarian stimulation treatment,
the data of the change in serum inhibin B level (ΔinhibinB) acquired refers to a difference between inhibin B concentrations in the venous blood of the subject on the 6th day and the 2nd day of the menstrual period after receiving the ovarian stimulation treatment,
the data of the basal follicle-stimulating hormone (basal FSH) level acquired refers to a concentration of FSH in the venous blood of the subject on the 2nd day of the menstrual period after receiving the ovarian stimulation treatment,
the data of the basal androstenedione (basal AND) level acquired refers to a concentration of androstenedione in the venous blood of the subject on the 2nd day of the menstrual period after receiving the ovarian stimulation treatment, and the data of the change in androstenedione level (ΔAND) refers to a difference between the concentrations of androstenedione in the venous blood of the subject on the 6th day and the 2nd day of the menstrual period after receiving the ovarian stimulation treatment, or
the data of the basal luteinizing hormone (basal LH) level acquired refers to a concentration of LH in the venous blood of the subject on the 2nd day of the menstrual period after receiving the ovarian stimulation treatment.

16. The method according to any one of items 11 to 15, wherein,
in the step for calculating a probability of high ovarian response, the formula I for calculating the probability of high ovarian response probability of the subject, which is obtained by fitting based on the data of the basal anti-Mullerian hormone (basal AMH) level and the change in serum inhibin B level (ΔinhibinB) of the patient receiving the standard ovarian stimulation treatment in the existing database, is a calculation formula which is obtained by fitting data of the anti-Mullerian hormone (AMH) level and change in INHBB level (ΔinhibinB) of the patient receiving the standard ovarian stimulation treatment in the existing database using logistic regression,
the formula I can be used to calculate the probability of high ovarian response of the subject by using the data of the basal anti-Mullerian hormone (basalAMH) level and dynamic change in inhibin B level (ΔinhibinB) of the subject acquired by the data collection module.

17. The method according to any one of items 14 to 16, wherein,
in the step for calculating a probability of high ovarian response, the formula II for calculating the probability of high ovarian response of the subject, which is obtained by fitting based on the data of the the basal anti-Mullerian hormone (basal AMH) level, the change in inhibin B level (ΔinhibinB), the basal follicle-stimulating hormone (basalFSH) level, the basal androstenedione (basal AND) level and the change therein (ΔAND), and the basal luteinizing hormone (basal LH) level of the patient receiving the standard ovarian stimulation treatment in the existing database, is a calculation formula obtained by fitting the data of the basal anti-Mullerian hormone (basal AMH) level, the change in inhibin level (ΔinhibinB), the basal follicle-stimulating hormone (basal FSH) level, the basal androstenedione (basal AND) level and the change therein (ΔAND), and the basal luteinizing hormone (basal LH) level of the patient receiving the standard ovarian stimulation treatment in the existing database using logistic regression;
the formula II can be used to calculate the probability of high ovarian response of the subject by using the data of the basal anti-Mullerian hormone (basalAMH) level, the change in inhibin B level (ΔinhibinB), the basal follicle-stimulating hormone (basal FSH) level, the basal androstenedione (basal AND) level and the change therein (ΔAND), and the basal luteinizing hormone (basal LH) level of the subject acquired by the data collection module.

18. The method according to item 16, wherein,
the formula I is:

$$P = 1 / ( 1 + {e}^{( - ( a + b * ln ( basal AMH ) + c * ln ( Δ inhibinB ) ) )} ) ,$$

wherein
p is a parameter calculated to characterize the probability of high ovarian response of the subject;
a is any value selected from a range of −18.49449 to −9.790517, preferably −14.1425;
b is any value selected from a range of 0.4227336 to 1.548814, preferably 0.9857738;
c is any value selected from a range of 0.9233156 to 2.2649428, preferably 1.5941292; and
e is a natural number.

19. The method according to claim 17, wherein,
the formula II is:

$$P = 1 / ( 1 + {e}^{( - ( d + f * basal FSH + g * ln ( basal AMH ) + h * basal AND + i * ln ( Δ inhibinB ) + j * Δ AND + k * basal LH ) )} ) ,$$

wherein
p is a parameter calculated to characterize the probability of high ovarian response of the subject,
d is any value selected from a range of −15.39228 to −6.729412, preferably −11.06085;
f is any value selected from a range of −0.589993 to −0.09121, preferably −0.340602;
g is any value selected from a range of 0.1606959 to 1.369763, preferably 0.7652294;
h is any value selected from a range of −0.013212 to 0.1226672, preferably 0.0547278;
i is any value selected from a range of 0.65715 to 2.0135819, preferably 1.3353659;
j is any value selected from a range of −0.036413 to 0.1330693, preferably 0.0483282;
k is any value selected from a range of −0.051137 to 0.2291053, preferably 0.088984; and
e is a natural number.

20. The method according to any one of items 14 to 19, wherein,
the step for calculating a probability of high ovarian response also comprises a comparison substep, in which probability values of high ovarian response of the subject calculated by formula I and formula II respectively are compared, and a larger probability value is taken as the probability of high ovarian response of the subject.

### Effects of the Invention

In the present application, firstly, the system for predicting high ovarian response of a subject of the present application can be used to predict the probability of high ovarian response of the subject. By using the system of the present application, the probability of high ovarian response can be predicted, so that patients with the risk of high ovarian response can be identified at the beginning of ovarian stimulation treatment, and effective measures can be taken to prevent such excessive response. For example, corresponding adjustments to drug dosage and regimen are made. The system and method of the present application utilize the dynamic change in serum inhibin B level as an evaluation index, and no use the AFC index with many defects in the prior art, thereby achieving the ability to accurately predict the probability of high ovarian response of the subject.

### BRIEF DESCRIPTION OF THE DRAWINGS

Various other advantages and benefits of the present application will become apparent to those of ordinary skill in the art by reading the following detailed description of the preferred specific embodiments. The drawings in the specification are only used for the purpose of illustrating the preferred embodiments and are not to be considered as limiting the present application. Obviously, the drawings described below are only some embodiments of the present application. For ordinary technicians in this field, other drawings can be acquired based on these drawings without any creative work. Also, throughout the drawings, the same reference numerals are used to denote the same components.

FIG. 1 shows the screening process for modeling (the black vertical line is the preferred model); wherein FIG. 1A shows the process of independent variables entering the model; after the black vertical line in FIG. 1B, if more independent variables enter the model to verify the data set, the effect will not be better, that is, the ordinate parameter will not continue to decrease, but will increase instead.

FIG. 2 shows comparison of AUC between Model 1 and Model 2.

FIG. 3 shows a relationship between predicted probability of high response and actual probability of occurrence of high response.

### DETAIL DESCRIPTION OF THE INVENTION

Specific embodiments of the present application will be described in more detail below with reference to the accompanying drawings. Although specific embodiments of the present application are shown in the drawings, it should be understood that the present application can be embodied in various forms and should not be limited to the embodiments set forth herein. On the contrary, these embodiments are provided to enable a more thorough understanding of the present application and to fully convey the scope of the present application to those skilled in the art.

It should be noted that certain terms are used in the specification and claims to refer to specific components. Those skilled in the art should understand that they may use different terms to refer to the same component. This specification and claims do not use differences in nouns as a way to distinguish components, but use differences in functions of components as the criterion for distinction. As mentioned throughout the specification and claims, “comprising” or “including” is an open-ended term and should be interpreted as “including but not limited to”. The following description of the specification is a preferred embodiment of the present application, but the description is for the purpose of general principles of the specification and is not intended to limit the scope of the present application. The protection scope of the present application shall be determined by the appended claims.

Several factors of infertility referred to in this application are defined below. Endometriosis is a common gynecological disease in women caused by the implantation of active endometrial cells outside the endometrium. Endometrial cells should grow in the uterine cavity. However, since the uterine cavity is connected to the pelvic cavity through the fallopian tube, endometrial cells can enter the pelvic cavity through the fallopian tube and grow ectopically. The main pathological changes of endometriosis are periodic bleeding of ectopic endometrium and fibrosis of surrounding tissues, forming ectopic nodules. Dysmenorrhea, chronic pelvic pain, menstrual abnormalities and infertility are its main symptoms. The lesions can affect all pelvic tissues and organs, and are most common in the ovaries, rectouterine pouch, and uterosacral ligaments, and can also occur in the abdominal cavity, thoracic cavity, limbs, etc. Tubal infertility refers to that the blockage or dysfunction of the fallopian tubes becomes the main cause of female infertility because the fallopian tubes have the important role of transporting sperm, picking up eggs and transporting fertilized eggs to the uterine cavity. The causes of blockage or dysfunction of fallopian tube are acute or chronic fallopian tube inflammation. In addition, unexplained infertility was defined as couples with a history of recurrent conception failure despite normal results of standard tests such as ovulation testing, tubal patency, and semen analysis.

Variable type: In statistics, variable types can be divided into quantitative variables and qualitative variables (also called categorical variables).

Quantitative variables are variables used to describe the quantity and number of things, and can be divided into continuous and discrete types. A continuous variable is a variable that can take any value within a certain interval. Its value is continuous and can have decimal points. For example, blood pressure values, blood sugar values, anthropometric height, weight, chest circumference, etc. are continuous variables, and their values can only be acquired by measurement or metering methods. A discrete variable is a variable whose value can only be a natural number or an integer unit. For example, pain scores, the number of metastatic lesions, the number of retrieved oocytes, etc., can only be positive numbers and cannot have decimal points. The values of such variables are generally acquired using counting methods.

The variable type is not static. Conversion can be performed between various variables according to the needs of the research purpose. For example, the hemoglobin level (g/L) is originally a numerical variable. If it is divided into two categories, normal and low hemoglobin, it can be analyzed as binomial data. If it is divided into five ranks, severe anemia, moderate anemia, mild anemia, normal, and increased hemoglobin, it can be analyzed as ranked data. Sometimes categorical data can also be quantified, for example, the patient's nausea reaction can be represented by 0, 1, 2, or 3, and it can be analyzed as numerical variable data (quantitative data).

Logistic regression is a generalized linear regression analysis model. The dependent variable of logistic regression can be binary or multi-classified, but binary classification is more commonly used and easier to explain. Multi-classification can be processed using the softmax method. The most commonly used one in practice is the binary logistic regression.

In the present application, the number of retrieved oocytes (NROs), refers to the number of oocytes with larger than 10 mm in diameter of follicles acquired during the ovarian stimulation after the subject receives ovarian stimulation treatment. The number of retrieved oocytes can be determined in a manner known to those skilled in the art. The detection of the number of retrieved oocytesin the present application is as follows: under ovarian stimulation protocol with the standard GnRH antagonist, human recombinant FSH (human rFSH) (e.g., Gonal-F alfa [Merck Serono, Germany], Puregon beta [MSD, USA], Urofollitropin [Livzon Pharmaceutical Group Inc., China] or Menotrophins [Livzon Pharmaceutical] Group Inc., China]) is administered from the 2nd day of the menstrual period. The starting dose of human rFSH is selected based on age, basal AMH level, basal FSH level, basal AFC level, and BMI. The rFSH dose was further adjusted based on the size and number of growing follicles observed by ultrasound and serum E₂ levels monitored during ovarian stimulation. When the growing follicles reach 10-12 mm in diameter, GnRH antagonist therapy is initiated. When at least two dominant follicles were observed to be more than 18 mm in diameter by ultrasound, hCG (Choriogonadotropin alfa, Merck Serono) was injected at a dose of 5000-10000 IU to trigger final oocyte maturation. The number of oocytes were measured 36-38 hours after hCG administration.

In the present application, the diagnostic criterion for high ovarian response is that the number of follicles with a diameter of more than 10 mm acquired during ovarian hyperstimulation is greater than 20.

Common indicators for predicting high ovarian response are as follows:
1) age, wherein most high-responders are young women, generally less than 35 years old;
2) antral follicle count (AFC) (number, size, uniformity) of the ovary, wherein generally, people with an AFC >20 are considered high responders:
3) level of hormone (basal follicle-stimulating hormone (bFSH), anti-Mullerian hormone (AMH), inhibin B), wherein when anti-Mullerian hormone (AMH) is greater than 4.5 mg/L, it is predicted that both false positive and false negative rates of the high response are low;
4) menstrual cycle length, wherein those with long and rare menstrual cycle are more likely to have high response; some researchers have designed a model to predict high response, and the results show that the accuracy of predicting high response by combining antral follicle count (AFC) with menstrual cycle is higher;
5) response to ovulation-inducing drugs, wherein in previous ovulation-inducing cycles, multiple follicles (>15 follicles with a diameter of 12-14 mm) developed, or the number of oocytes collected was >18, or OHSS occurred in the past.

However, most of the above indicators are judged based on the clinical experience of clinicians. This judgment method is inaccurate, mainly relying on subjective experience, and there is no unified standard.

To this end, the present application provides a system for predicting high ovarian response of a subject, which comprises: a data collection module, which is used for acquiring data of basic anti-mullerian hormone (AMH) level and serum inhibin B (inhibinB) level; and a module for calculating a probability of high ovarian response, which is used for performing calculation on the above data acquired in the data collection module, so as to calculate the probability of high ovarian response of the subject, to prevent ovarian overstimulation, and reduce the cost during ovarian stimulation.

In the present application, the subject is a subject who will receive standard ovarian stimulation treatment.

In the present application, serum anti-Mullerian hormone (AMH) refers to a hormone secreted by the granulosa cells of the small ovarian follicles. Female babies begin to produce AMH during the fetal period. The more small follicles there are in the ovaries, the higher the concentration of AMH. Conversely, as the follicles are gradually consumed with age and various factors, the AMH concentration will also decrease. As menopause approaches, AMH gradually approaches 0.

In the present application, serum inhibin B level is considered as a marker of follicular development. Inhibin B participates in the selection of follicles in the normal menstrual period through endocrine and paracrine effects and promotes the growth of follicles. One of the effects of inhibin B is to downregulate FSH secretion during the mid-follicular phase of the natural menstrual period. It also exerts a paracrine effect, stimulating the production of androgens and LH by the ootheca cells. The secretion of inhibin B reaches its peak in in early stages of follicles, with a diameter of 10-12 mm millimeters. It has been demonstrated that inhibin B at day 5 (early follicular phase) is a superior marker of poor ovarian response and live birth compared to basal markers. Inhibin B is produced primarily by FSH-sensitive follicles, and administration of exogenous FSH leads to its increase in growing follicles. Consistent with this, the inventors of the present application found that the dynamic change in inhibin B level (ΔINHB), i.e., a difference between the inhibin B concentrations on the 6th day and the 2nd day of the menstruation in a cycle of ovulation induction treatment, is the best marker for predicting the number of oocytes retrieved.

In the present application, there is no limitation on the data collection module, as long as it can be used to obtain the data of basal anti-Mullerian hormone (AMH) level and data of change in serum inhibin B (inhibinB) level of the subject, wherein, specifically, the data of basal anti-Mullerian hormone (basalAMH) level acquired refers to a concentration of the anti-Mullerian hormone in the venous blood of the subject on the 2nd day of the menstrual period after receiving ovarian stimulation treatment; the data of the change in serum inhibin B level (ΔinhibinB) acquired refers to a difference between the inhibin B concentrations in the venous blood of the subject on the 6th day and the 2nd day of the menstrual period after receiving ovarian stimulation treatment.

In the present application, a module for calculating a probability of high ovarian response is used to performing calculation on the above data (the data of basic anti-Mullerian hormone (AMH) level, and the data of the change in serum inhibin B level (ΔinhibinB) of the subject) acquired by the data collection module, so as to calculate the probability of high ovarian response of the subject. First of all, it should be understood that, in this module, formula I for calculating the probability of high ovarian response of the subject is pre-stored, wherein the formula I is obtained by fitting based on data of basal anti-Mullerian hormone (basal AMH) level and change in serum inhibin B level (ΔinhibinB) of a patient receiving standard ovarian stimulation treatment in an existing database By using such pre-stored formula, calculations can be performed on the date of basic anti-Mullerian hormone (AMH) level and change in serum inhibin B level (ΔinhibinB) of any subject acquired through the data collection module.

Specifically, this pre-stored formula is obtained by using logistic regression to fit the pre-stored data of basic anti-Mullerian hormone (AMH) level and change in serum inhibin B level (ΔinhibinB) of the patient receiving standard ovarian stimulation treatment in an existing database. During the calculation, the data of basic anti-Mullerian hormone (AMH) level and change in serum inhibin B level (ΔinhibinB) acquired by the data collection module for the subject to be tested are brought into the above formula to calculate the probability of high ovarian response of the subject to be tested.

Furthermore, the inventors of the present application have constructed a specific formula for predicting the probability of high ovarian response. When the data collection module acquires the data of basic anti-Mullerian hormone (AMH) level and change in serum inhibin B level (ΔinhibinB), the specific formula is the following formula I:

$$P = 1 / ( 1 + {e}^{( - ( a + b * ln ( basal AMH ) + c * ln ( Δ inhibinB ) ) )} ) \qquad ( Formula 1 )$$

Furthermore, in the formula I,
wherein,
p is a parameter calculated to characterize the probability of high ovarian response of the subject;
a is any value selected from −18.49449~−9.790517, preferably −14.1425;
b is any value selected from 0.4227336~1.548814, preferably 0.9857738;
c is any value selected from 0.9233156~2.2649428, preferably 1.5941292;
e is a natural number.

In some embodiments of the present application, in addition to obtaining the data of the basal anti-Mullerian hormone (AMH) level and serum inhibin B (inhibinB) level of the subject, the data collection module further obtains the follicle-stimulating hormone (FSH) level, androstenedione (AND) level and luteinizing hormone (LH) level of the subject.

In the present application, follicle-stimulating hormone (FSH) refers to a hormone secreted by basophils of the anterior pituitary gland, which is composed of glycoprotein and mainly functions to promote follicle maturation. FSH can promote the proliferation and differentiation of granulosa cells of the follicle and promote the growth of the entire ovary. Its action on the seminiferous tubules of the testicles can promote sperm formation. FSH is secreted in a pulsatile manner in the human body, and in women it changes with the menstrual cycle. The determination of FSH in serum is of great significance for the diagnosis and treatment of infertility and endocrine diseases, such as understanding the pituitary endocrine function, indirectly understanding the functional status of the ovaries, evaluating the ovarian reserve and ovarian responsiveness, and formulating the dosage of ovarian stimulation drugs.

In the present application, androstenedione (AND) is one of the four male hormones in female blood circulation. Androstenedione is the main precursor of testosterone. The androstenedione in the circulation is secreted equally by the ovaries and adrenal glands. There is a dynamic equilibrium relationship between the androstenedione level and the testosterone level. Too high concentration of androstenedione in circulating blood indicates hyperandrogenism.

In the present application, the change in androstenedione level (ΔAND) refers to data of change in the androstenedione level, that is, the difference between the androstenedione concentrations in the venous blood of the subject on the 6th day and the 2nd day of the menstrual period after receiving ovarian stimulation treatment.

In the present application, luteinizing hormone (LH) is a glycoprotein gonadotropin secreted by pituitary cells, and can promote the conversion of cholesterol into sex hormones in gonadal cells. For women, it works together with follicle-stimulating hormone (FSH) to promote follicle maturation, estrogen secretion, ovulation, and the formation and maintenance of the corpus luteum, secreting progesterone and estrogen. For men, luteinizing hormone promotes the synthesis and release of testosterone by testicular interstitial cells. LH level refers to a concentration of the LH in venous blood serum sample of a female subject on days 2-4 of menstruation.

In the present application, there is no limitation on the data collection module, as long as it can be used to acquire the data of the basal anti-Mullerian hormone (AMH) level, the change in serum inhibin B (inhibinB) level, the basal follicle-stimulating hormone (basal FSH) level, the basal androstenedione (basal AND) level and the change therein h (ΔAND), and basal luteinizing hormone (basal LH) level of the subject. Specifically, the data of basal anti-Mullerian hormone (basal AMH) level acquired refers to a concentration of anti-Mullerian hormone in the venous blood of the subject on the 2nd day of the menstrual period after receiving the ovarian stimulation treatment; the data of the change in serum inhibin B level (ΔinhibinB) acquired refers to a difference between inhibin B concentrations in the venous blood of the subject on the 6th day and the 2nd day of the menstrual period after receiving the ovarian stimulation treatment, the data of the basal follicle-stimulating hormone (basal FSH) level acquired refers to a concentration of FSH in the venous blood of the subject on the 2nd day of the menstrual period after receiving the ovarian stimulation treatment the data of the basal androstenedione (basal AND) level acquired refers to a concentration of androstenedione in the venous blood of the subject on the 2nd day of the menstrual period after receiving the ovarian stimulation treatment, and the data of the change in androstenedione level (ΔAND) refers to a difference between the concentrations of androstenedione in the venous blood of the subject on the 6th day and the 2nd day of the menstrual period after receiving the ovarian stimulation treatment, or the data of the basal luteinizing hormone (basal LH) level acquired refers to LH concentration in the venous blood of the subject on the 2nd day of the menstrual period after receiving the ovarian stimulation treatment.

In the present application, the module for calculating a probability of high ovarian response is used to perform calculation on the above data (the data of the basal anti-Mullerian hormone (AMH) level, the change in serum inhibin B level (ΔinhibinB), the basal follicle-stimulating hormone (basal FSH) level, the basal androstenedione (basal AND) level and the change therein (ΔAND), and the basal luteinizing hormone (basal LH) level of the subject) acquired by the data collection module, so as to calculate the probability of high ovarian response of the subject. First, it should be understood that in this module, the data of the basal anti-Mullerian hormone (AMH) level, the change in serum inhibin B level (ΔinhibinB), the basal follicle-stimulating hormone (basal FSH) level, the basal androstenedione (basal AND) level and the change therein (ΔAND), and the basal luteinizing hormone (basal LH) level of the patient receiving standard ovarian stimulation treatment in an existing database, and the formula obtained by fitting based on the pre-stored data of the patient are pre-stored. By using such the pre-stored formula, calculation can be performed on the data of the basal anti-Mullerian hormone (AMH) level, the change in serum inhibin B level (ΔinhibinB), the basal follicle-stimulating hormone (basal FSH) level, the basal androstenedione (basal AND) level and the change therein (ΔAND), and the basal luteinizing hormone (basal LH) level of any subject acquired by the data collection module.

Specifically, the pre-stored formula is obtained by fitting the data of the basal anti-Mullerian hormone (basal AMH) level, the change in inhibin level (ΔinhibinB), the basal follicle-stimulating hormone (basal FSH) level, the basal androstenedione (basal AND) level and the change therein (ΔAND), and the basal luteinizing hormone (basal LH) level of the patient receiving the standard ovarian stimulation treatment in the existing database by logistic regression.

During the calculation, the data of the basal anti-Mullerian hormone (basal AMH) level, the change in inhibin level (ΔinhibinB), the basal follicle-stimulating hormone (basal FSH) level, the basal androstenedione (basal AND) level and the change therein (ΔAND), and the basal luteinizing hormone (basal LH) level of acquired by the data collection module for the subject to be tested is brought into the above formula to calculate the probability of high ovarian response of the subject to be tested.

Furthermore, the inventors of the present application have constructed a specific formula for predicting the probability of high ovarian response. When the data collection module acquires the data of the basal anti-Mullerian hormone (basal AMH) level, the change in inhibin level (ΔinhibinB), the basal follicle-stimulating hormone (basal FSH) level, the basal androstenedione (basal AND) level and the change therein (ΔAND), and the basal luteinizing hormone (basal LH) level, the specific formula is the following formula II:

$$P = 1 / ( 1 + {e}^{( - ( d + f * basal FSH + g * ln ( basal AMH ) + h * basal AND + i * ln ( Δ inhibinB ) + j * Δ AND + k * basal LH ) )} ) \qquad ( Formula II )$$

Furthermore, in the Formula II,
wherein,
p is a parameter calculated to characterize the probability of high ovarian response of the subject,
d is any value selected from −15.39228~−6.729412, preferably −11.06085;
f is any value selected from −0.589993~−0.09121, preferably −0.340602;
g is any value selected from 0.1606959~1.369763, preferably 0.7652294;
h is any value selected from −0.013212~0.1226672, preferably 0.0547278;
i is any value selected from 0.65715~2.0135819, preferably 1.3353659;
j is any value selected from −0.036413~0.1330693, preferably 0.0483282;
k is any value selected from −0.051137~0.2291053, preferably 0.088984;
e is a natural number.

In some embodiments of the present application, the module for calculating a probability of high ovarian response also comprises a comparison submodule, in which probability values of high ovarian response of the subject calculated by formula I and formula II respectively are compared, and a larger probability value is taken as the probability of high ovarian response of the subject.

The present application also relates to a method for using the above system to predict high ovarian response of a subject, comprising:
a data collection step, in which data of basal anti-Mullerian hormone (AMH) level and change in serum inhibin B level of the subject is acquired; and
a step for calculating a probability of high ovarian response, in which calculation is performed on the data acquired in the data collection step, so as to calculate the probability of high ovarian response of the subject.

In the above method, follicle-stimulating hormone (FSH) level, basal androstenedione (basal AND) level and change therein (AND), and basal luteinizing hormone (LH) level of the subject are further acquired; the subject is a subject who will receive standard ovarian stimulation treatment.

In the above method, in the step for calculating a probability of high ovarian response, formula I for calculating the probability of high ovarian response of the subject is pre-stored for use in this step, wherein the formula I is obtained by fitting based on data of basal anti-Mullerian hormone (basal AMH) level and change in serum inhibin B level (ΔinhibinB) of a patient receiving standard ovarian stimulation treatment in an existing database.

In the above method, in the step for calculating a probability of high ovarian response, formula II for calculating the probability of high ovarian response of the subject is pre-stored for use, wherein the formula II is obtained by fitting based on data of basal anti-Mullerian hormone (basal AMH) level, change in serum inhibin B level (ΔinhibinB), basal follicle-stimulating hormone (basal FSH) level, basal androstenedione (basal AND) level and change therein (ΔAND), and basal luteinizing hormone (basal LH) level of a patient receiving the standard ovarian stimulation treatment in an existing database.

In the above method, in the data collection step, the data of the basal anti-Mullerian hormone (basalAMH) level acquired refers to concentration of anti-Mullerian hormone in venous blood of the subject on the 2nd day of menstrual period after receiving the ovarian stimulation treatment.

In the above method, in the data collection step, the data of the change in serum inhibin B level (ΔinhibinB) acquired refers to a difference between inhibin B concentrations in the venous blood of the subject on the 6th day and the 2nd day of the menstrual period after receiving the ovarian stimulation treatment.

In the above method, in the data collection step, the data of the basal follicle-stimulating hormone (basal FSH) level acquired refers to a concentration of FSH in the venous blood of the subject on the 2nd day of the menstrual period after receiving the ovarian stimulation treatment.

In the above method, in the data collection step, the data of the basal androstenedione (basal AND) level acquired refers to a concentration of androstenedione in the venous blood of the subject on the 2nd day of the menstrual period after receiving the ovarian stimulation treatment, and the data of the change in androstenedione level change (ΔAND) refers to a difference between the concentrations of androstenedione in the venous blood of the subject on the 6th day and the 2nd day of the menstrual period after receiving the ovarian stimulation treatment.

In the above method, in the data collection step, the data of the basal luteinizing hormone (basal LH) level acquired refers to a concentration of LH in the venous blood of the subject on the 2nd day of the menstrual period after receiving the ovarian stimulation treatment.

In the above method, in the step for calculating a probability of high ovarian response, formula I for calculating the probability of high ovarian response probability of the subject, which is obtained by fitting based on the data of the basal anti-Mullerian hormone (basal AMH) level and the change in serum inhibin B level (ΔinhibinB) of the patient receiving the standard ovarian stimulation treatment in the existing database, is a calculation formula obtained by fitting data of the anti-Mullerian hormone (AMH) level and change in INHBB level (ΔinhibinB) of the patient receiving the standard ovarian stimulation treatment in the existing database by logistic regression, the formula I can be used to calculate the probability of high ovarian response occurrence of the subject to be tested by using the data of the basal anti-Mullerian hormone (basal AMH) level and dynamic change in inhibin B level (ΔinhibinB) of the subject acquired in the data collection step.

In the above method, in the step for calculating a probability of high ovarian response, the formula II for calculating the probability of high ovarian response of the subject, which is obtained by fitting based on the data of the the basal anti-Mullerian hormone (basal AMH) level, the change in inhibin B level (ΔinhibinB), the basal follicle-stimulating hormone (basalFSH) level, the basal androstenedione (basal AND) level and the change therein (ΔAND), and the basal luteinizing hormone (basal LH) level of the patient receiving the standard ovarian stimulation treatment in the existing database, is a calculation formula obtained by fitting the data of the basal anti-Mullerian hormone (basal AMH) level, the change in inhibin level (ΔinhibinB), the basal follicle-stimulating hormone (basal FSH) level, the basal androstenedione (basal AND) level and the change therein (ΔAND), and the basal luteinizing hormone (basal LH) level of the patient receiving the standard ovarian stimulation treatment in the existing database by logistic regression;
the formula II can be used to calculate the probability of high ovarian response of the subject to be tested by using the data of the basal anti-Mullerian hormone (basal AMH) level, the change in inhibin B level (ΔinhibinB), the basal follicle-stimulating hormone (basal FSH) level, the basal androstenedione (basal AND) level and the change therein (ΔAND), and the basal luteinizing hormone (basal LH) level of the subject to be tested acquired in the data collection step.

In the above method, the formula I is:

$$P = 1 / ( 1 + {e}^{( - ( a + {b}^{*} ln ( basal AMH ) + c * ln ( Δ inhibinB ) ) )} )$$

wherein,
p is a parameter calculated to characterize the probability of high ovarian response of the subject;
a is any value selected from −18.49449~−9.790517, preferably −14.1425;
b is any value selected from 0.4227336~1.548814, preferably 0.9857738;
c is any value selected from 0.9233156~2.2649428, preferably 1.5941292;
e is a natural number.

In the above method, the formula II is:

$$P = 1 / ( 1 + {e}^{( - ( d + f * basal FSH + g * l n ( basal AMH ) + h * basal AND + i * ln ( Δ inhibinB ) + j * Δ AND + k * basal LH ) )} )$$

wherein,
p is a parameter calculated to characterize the probability high ovarian response of the subject,
d is any value selected from −15.39228~−6.729412, preferably −11.06085;
f is any value selected from −0.589993~−0.09121, preferably −0.340602;
g is any value selected from 0.1606959~1.369763, preferably 0.7652294;
h is any value selected from −0.013212~0.1226672, preferably 0.0547278;
i is any value selected from 0.65715~2.0135819, preferably 1.3353659;
j is any value selected from −0.036413~0.1330693, preferably 0.0483282;
k is any value selected from −0.051137~0.2291053, preferably 0.088984;
e is a natural number.

In the above method, the step for calculating a probability of high ovarian response also comprises a comparison substep, in which probability values of the high ovarian response of the subject calculated by formula I and formula II respectively are compared, and a larger probability value is taken as the probability of high ovarian response of the subject.

the step for calculating probability of high ovarian response also comprises a comparison substep, which compares the high ovarian response occurrence probability values of the subject calculated by Formula I and Formula II respectively, and takes the larger probability value as the high ovarian response occurrence probability of the subject.

### Examples

### 1. Subjects Used to Confirm Construction of the Model

A model was preliminarily constructed based on data of 669 patients treated at Peking University Third Hospital between January 2018 and June 2020. For the patients used for preliminary model construction, the basic and clinical characteristics of the patients were collected, including surname, medical record number, serial number, age, menstrual cycle, response to ovarian stimulation drugs, BMI index, duration of infertility, attempts of previous in vitro fertilization/intracytoplasmic sperm injection-embryo transfer (IVF/ICSI-ET), AMH levels on the 2nd day and the 6th day of menstrual in an ovarian stimulation cycle, inhibin B levels and basal FSH levels on the 2nd day and the 6th day of menstrual in an ovarian stimulation cycle, AND levels on the 2nd day and the 6th day of menstrual and change in AND level in an ovarian stimulation cycle, serum testosterone (T) levels on the 2nd day and the 6th day of menstrual in an ovarian stimulation cycle, LH levels on the 2nd day and the 6th day of menstrual in an ovarian stimulation cycle, serum E₂ levels on the 2nd day and the 6th day of menstrual in an ovarian stimulation cycle, AFCs of the left and right ovaries, the first, second, third, fourth and fifth causes of infertility, conventional or mild ovarian stimulation cycles, type of ovarian stimulation/COS regimen, starting dose and total dose of recombinant rFSH, duration of rFSH treatment (days), name of rFSH, endometrial thickness on the day of human chorionic gonadotropin (hCG) triggering, date of oocyte retrieval and number of retrieved oocytes.

The serum FSH, AFC, AND, ΔAND, LH, E₂, T and AND measurements were all performed using the Siemens Immulite 2000 immunoassay system (Siemens Healthcare Diagnostics, Shanghai, PR China). Quality controls for these assays were provided by Bio-RAD Laboratories (Lyphochek Immunoassay Plus Control, Trilevel, Catalog No. 370, Batch No. 40340).

Serum AMH concentrations and inhibin B concentrations were measured using ultrasensitive ELISA kits (Ansh Laboratories, Webster, TX, USA) with quality controls provided by the kits. The coefficient of variation of the assays for three-level or two-level control was less than 5% for AMH, inhibin B, FSH, and LH, respectively. For E₂, T and AND, the coefficient of variation of the assays for three-level or two-level controls were less than 10%, respectively. The results were shown in Table 1, which show the ovarian reserve index for the basal level (day 2) and the change level (value of day 6 minus value of day 2) in the cycle with conventional GnRH antagonist.

The present application performs the following detection on the number of retrieved oocytes (or the number of oocytes) of subjects who will receive the standard ovarian stimulation treatment: under the standard ovarian stimulation protocol with GnRH antagonist, human recombinant FSH (human rFSH) (e.g., Gonal-F alfa [Merck Serono, Germany], Puregon beta [MSD, USA], Urofollitropin [Livzon Pharmaceutical Group Inc., China] or Menotrophins [Livzon Pharmaceutical] Group Inc., China]) was administered from the 2nd day of the menstrual cycle. Doctors choose the starting dose of human rFSH according to experience, mainly based on age, basal AMH level, basal FSH level, basal AFC level, and BMI. The rFSH dose was further adjusted based on the size and number of growing follicles observed by ultrasound and monitoring of serum E₂ levels during ovarian stimulation. When the growing follicles reached 10-12 mm in diameter, GnRH antagonist therapy was initiated. When at least two dominant follicles were observed to be more than 18 mm in diameter by ultrasound, hCG (Choriogonadotropin alfa, Merck Serono) was injected at a dose of 5000-10000 IU to trigger final oocyte maturation. The number of oocytes were detected 36-38 hours after hCG administration.

### 2. Preliminary Screening of Indicators for Constructing Models

In this Example, the presence or absence of the high ovarian response was used as the dependent variable, and age, BMI, AFC, serum basal FSH level, serum AMH levels on the 2nd day and the 6th day, serum inhibin B levels on the 2nd day and the 6th day, serum LH levels on the 2nd day and the 6th day, serum estradiol (E₂) level on the 2nd day and the 6th day, serum testosterone level on the 2nd day and the 6th day, serum androstenedione (AND) level on the 2nd day and the 6th day, serum progesterone (P) level on the 2nd day and the 6th day, etiology and other indicators (a total of 15) were used as independent variables.

For the variable selection process, firstly, a part (70%) of the data collected in the above part 1 was randomly selected as the training set for model constructing, and the rest of the data (30%) was used as the test set, for model evaluation. Then a prediction model was built in the training set. The scaled negative log-likelihood (−Log L(β)) was used to evaluate the final model: the smaller the scaled −Log L(β) value in the validation set, the better the model fit. The logistic least absolute shrinkage and selection operator (LASSO) model was a shrinkage method, in which active selection can be made from a large and potentially multicollinear set of variables and the possibility of overfitting can be reduced, for constructing predictive models. Logistic LASSO was a logistic regression analysis method that penalizes the absolute size of the coefficients of the regression model according to the value of the penalty term λ. The larger the penalty, the closer the estimated value of weaker factors will be to zero, so only the strongest predictors are retained in the model. The value of λ was determined by 10-fold cross validation, and the most predictive covariates selected by the minimum value (λmin) were used to construct the PCOS diagnostic model. The performance of each model was evaluated using the area under curve (AUC) of the receiver operating characteristic, sensitivity and specificity, and 95% confidence intervals (CI). All analyses in this study were performed using SAS JMP Pro (version 14.2; SAS Institute, Cary, NC, USA), and p<0.05 was considered statistically significant.

Table 1 showed the results of the preliminary screening of indicators for constructing the model.

TABLE 1

| | Markers for ovarian reserve | |
|---|---|---|
| | the level onthe 2nd day | the level on the 6th day |
| Age (years) | 33(30-36) | NA |
| BMI (kg/m²) | 21.9 (20.0-24.5) | NA |
| FSH (IU/L) | 6.26 (5.16-7.93) | NA |
| LH (IU/L) | 3.43 (2.43-4.76) | −1.94(−2.99~−1.05) |
| E₂ (pmol/L) | 151 (121-176) | 1113 (474-2093) |
| AMH (ng/mL) | 3.02 (1.63-5.33) | −0.5 (−1.21~−0.16) |
| Inhibin B (pg/mL) | 87.9 (62.7-114.0) | 642 (309-1172) |
| T (nmol/L) | 0.69 (0.69-0.80) | 0 (0-0.15) |
| AND (nmol/L) | 6.58 (4.96-9.21) | 0.87(−0.74~2.87) |

Note:
Values are expressed as medians; Δ level change, dynamic level of various markers for ovarian reserve on day 6 minus that on day 2; NORs, number of retrieved oocytes; BMI, body mass index; T, testosterone; AND, androstenedione; NA, not applicable

Based on the above Logistic LASSO analysis, it was shown that age, BMI, and FSH were not effective in predicting high ovarian response, so these indicators were removed from the subsequent modeling process.

3. Construction of Model 1 (i.e. The Model Involved in Formula II of this Application)

The data set consisting of the above 669 patients was randomly divided into two parts, one as a training set (468 data, 70%), and the other as a validation set (201 data, 30%).

First, the model was constructed in the training set and the model effect was verified in the validation set. The selection of the prediction model was mainly based on the negative log-likelihood value in the validation set. The lower the negative log-likelihood value in the validation set, the better the model.

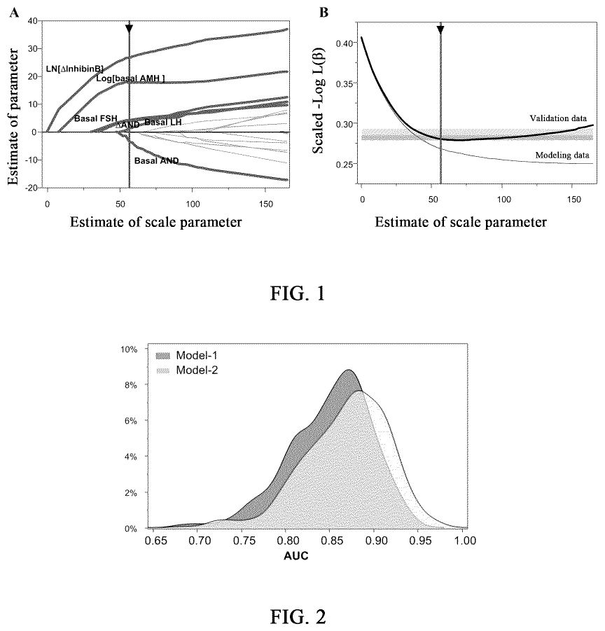

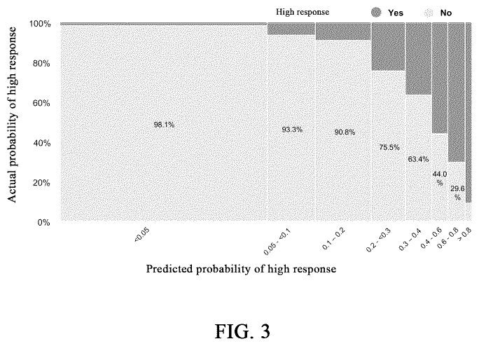

Model 1 (formula II) includes 6 variables, and the scaled −Log L(β) of the validation data part no longer decreases (−Log L(β) can be understood as the magnitude of the variance). The smaller the −Log L(β), the better. Therefore, the six variables, i.e., ln[ΔinhibinB], ln[basal AMH], basal AND, basal LH, basal FSH, and ΔAND, were finally included in the model according to their importance. As shown in FIG. 1, when six variables were included, the vertical axis of the validation data, scaled −Log L(β), enters the area above the dark gray line in FIG. 1B. At this time, the variables was successively ln[ΔinhibinB], ln[basal AMH(d2)], basal FSH, ΔAND, basal AND and basal LH according to the importance.

At this time, the parameter estimation results of each variable in the prediction model were shown in Table 2, and Table 2 further showed the 95% confidence interval of each parameter.

TABLE 2
Performance of model 1 on the training set and validation set

| | Training set | | | Validation set | | |
|---|---|---|---|---|---|---|
| Measure | Estimate | Lower 95% CI | Upper 95% CI | Estimate | Lower 95% CI | Upper 95% CI |
| AUC | 0.8961 | 0.8511 | 0.9286 | 0.8823 | 0.7877 | 0.9381 |
| Prevalence | 0.140 | 0.113 | 0.173 | 0.141 | 0.091 | 0.211 |
| Sensitivity | 0.431 | 0.323 | 0.546 | 0.389 | 0.203 | 0.614 |
| Specificity | 0.980 | 0.962 | 0.989 | 0.973 | 0.923 | 0.991 |
| PPV | 0.775 | 0.625 | 0.877 | 0.700 | 0.397 | 0.892 |
| NPV | 0.914 | 0.885 | 0.936 | 0.907 | 0.841 | 0.947 |
| Positive LR | 21.145 | 10.510 | 42.541 | 14.259 | 4.055 | 50.137 |
| Negative LR | 0.581 | 0.475 | 0.711 | 0.628 | 0.434 | 0.909 |

It can be seen from Table 2 that the data obtained from the training set was basically consistent with the data obtained from the validation set.

Based on the above method, the following formula II was confirmed in this example.

P=1/(1+e^{(-(d+f*basal FSH+g*ln(basal AMH)+h*basal AND+i*ln(ΔinhibinB)+j*ΔAND+k*basal LH))}) (Formula II)

AMH represented the basal anti-Mullerian hormone level of the subject before the ovarian stimulation treatment; ΔinhibinB represents the dynamic change of inhibin B level in the early stage of the ovarian stimulation treatment.

In a specific embodiment, AMH referred to the concentration of the anti-Mullerian hormone in the venous blood of the subject at any time point during the menstrual period before ovarian stimulation treatment. The data of change in serum inhibin B level (ΔinhibinB) referred to the difference between the inhibin B concentrations in the venous blood of the subject on the 6th day and the 2nd day of the menstrual period after receiving the ovarian stimulation treatment. The data of the basal FSH level referred to the FSH concentration in the venous blood of the subject on the 2nd day of the menstrual period after receiving the ovarian stimulation treatment. The data of the basal androstenedione (basal AND) level referred to the androstenedione concentration in the venous blood of the subject on the 2nd day of the menstrual period after receiving the ovarian stimulation treatment, and the data of the change in androstenedione level (ΔAND) referred to the difference between the androstenedione concentrations in the venous blood of the subject on the 6th day and the 2nd day of the menstrual period after receiving the ovarian stimulation treatment. The data of the basal LH level referred to the LH concentration in the venous blood of the subject on the 2nd day of the menstrual period after receiving the ovarian stimulation treatment.

In the above Formula II, p was a calculated parameter for characterizing the probability of high ovarian response of the subject, d was any value selected from −15.39228 to −6.729412, preferably −11.06085; f was any value selected from −0.589993 to −0.09121, preferably −0.340602; g was any value selected from 0.1606959 to 1.369763, preferably 0.7652294; h was any value selected from −0.013212 to 0.1226672, preferably 0.0547278; i was any value selected from 0.65715 to 2.0135819, preferably 1.3353659; j was any value selected from −0.036413 to 0.1330693, preferably 0.0483282; k was any value selected from −0.051137 to 0.2291053, preferably 0.088984, and e was a natural number.

4. Construction of Model 2 (i.e., the Model Involved in Formula I of this Application)

In model 1, the variable importance of the six parameters was compared. The variable importance reflects the impact of each variable on the outcome variation. The greater the impact, the greater the importance of the variable. The variable importance was mainly reflected by main effects and total effects. The main effect reflects the influence of each variable itself on the outcome variation, and the total effect reflects the influence of each variable combined with other variables on the outcome variation. The results of variable importance are shown in Table 3. In this application, variable importance was based on the results calculated by SAS JMP Pro.

TABLE 3

| Column | Main effect | Total effect |
|---|---|---|
| ln[ΔinhibinB] | 0.566 | 0.692 |
| ln[basal AMH] | 0.221 | 0.333 |
| Basal FSH | 0.018 | 0.041 |
| \| ΔAND | 0.017 | 0.033 |
| Basal AND | 0.014 | 0.03 |
| Basal LH | 0.005 | 0.012 |
| Basal E₂ | 0.001 | 0.003 |
| Basal inhibinB | 0.001 | 0.002 |

As can be seen from Table 3, in model 1, the ln[ΔinhibinB] has the largest proportion of main effect, and explained 56.6% of the outcome variation, indicating that this indicator had the greatest impact on the outcome and was most important in predicting the probability of high ovarian response occurrence of the subject. The second was ln[basal AMH], which explained 22.1% of the outcome variation. The explanatory power of the remaining indicators was approximately 5% in total.

The applicant then established model 2 (Formula I) based on the two most important indicators, ΔinhibinB and basal AMH. In model 2 (Formula I), the data set consisting of the above 669 patients was randomly divided into two parts, one part as the training set (468 data, 70%), and the other part as the validation set (201 data, 30%).

First, the model was constructed in the training set and the model effect was verified in the validation set. The selection of the prediction model was mainly based on the negative log-likelihood value in the validation set. The lower the negative log-likelihood value in the validation set, the better the model.

The model 2 included two variables, and the scaled −Log L(β) no longer decreased, so the two variables which are ln[ΔinhibinB] and ln[basal AMH] were finally included in the model based on their importance. At this time, the parameter estimation results of each variable in the prediction model were shown in Table 4, which further showed the 95% confidence interval of each parameter.

TABLE 4
Performance of model 2 on the training set and validation set

| | Training set | | | Validation set | | |
|---|---|---|---|---|---|---|
| Measure | Estimate | Lower 95% CI | Upper 95% CI | Estimate | Lower 95% CI | Upper 95% CI |
| AUC | 0.8751 | 0.8273 | 0.9110 | 0.9035 | 0.8363 | 0.9450 |
| Prevalence | 0.140 | 0.113 | 0.173 | 0.141 | 0.091 | 0.211 |
| Sensitivity | 0.389 | 0.285 | 0.504 | 0.500 | 0.290 | 0.710 |
| Specificity | 0.984 | 0.968 | 0.992 | 0.927 | 0.863 | 0.963 |
| PPV | 0.800 | 0.641 | 0.900 | 0.529 | 0.310 | 0.738 |
| NPV | 0.908 | 0.879 | 0.931 | 0.919 | 0.853 | 0.957 |
| Positive LR | 24.556 | 11.146 | 54.100 | 6.875 | 3.054 | 15.479 |
| Negative LR | 0.621 | 0.516 | 0.747 | 0.539 | 0.339 | 0.858 |

It can be seen from Table 4 that the data obtained from the training set was basically consistent with the data obtained from the validation set.

In this Example, the following Formula I was confirmed.

P=1/(1+e^{(-(a+b*ln(basal AMH)+c*ln(ΔinhibinB)))}) (Formula I)

AMH represented the basal anti-Mullerian hormone level of the subject before the ovarian stimulation treatment; ΔinhibinB represented the change in inhibin B level in the early stage of ovarian stimulation treatment.

In a specific embodiment, AMH referred to a concentration of the anti-Mullerian hormone in the venous blood of the subject at any time point during the menstrual period before the ovarian stimulation treatment. FSH referred to a concentration of the follicle-stimulating hormone in the venous blood of female subject on the 2nd day of the menstrual before the ovarian stimulation treatment. The data of change in serum inhibin B level (ΔinhibinB) referred to the difference between the inhibin B concentrations in the venous blood of the subjects on the 6th day and the 2nd day of the menstrual period after receiving the ovarian stimulation treatment.

In the Formula I,
p was a parameter calculated to characterize the probability of high ovarian response of the subject;
a was any value selected from −18.49449~−9.790517, preferably −14.1425;
b was any value selected from 0.4227336~1.548814, preferably 0.9857738;
c was any value selected from 0.9233156~2.2649428, preferably 1.5941292;
e was a natural number.

### Comparison Between Model 1 and Model 2

For the AUC of ROC curve of the validation set of Model 1 and the validation set of Model 2, the bootstrap sampling was repeated 1000 times. The sampling distribution of the first model and the second model was shown in FIG. 2. It can be seen that the difference in AUC between two models was very small, but model 1 was more concise. Therefore, in practical applications, the specific model can be flexibly selected according to the situation of various clinical indicators.

If conditions permit, the calculations were performed for two models, and the larger calculated probability value can be taken as the probability of high ovarian response of the subject, which can further improve the accuracy of the prediction.

Although the embodiments of the present application are described above in conjunction with the accompanying drawings, the present application was not limited to the above-mentioned specific embodiments and application fields. The above-mentioned specific embodiments are merely illustrative and guiding, but not restrictive. Under the inspiration of this specification and without departing from the scope of protection of the claims of the present application, ordinary technicians in this field can also make many forms, which are all protected by the present application.

1-10. (canceled)

11. A method for predicting high ovarian response in a subject, comprising:
a data collection step, in which data of basal anti-Mullerian hormone (AMH) level and serum inhibin B level of the subject is acquired; and
a step for calculating a probability of high ovarian response, in which calculation is performed on the data acquired in the data collection step, so as to calculate the probability of high ovarian response of the subject.

12. The method according to claim 11, wherein
in the data collection step, follicle-stimulating hormone (FSH) level, androstenedione (AND) level, and luteinizing hormone (LH) level of the subject are further acquired;
the subject is a subject who will receive standard ovarian stimulation treatment.

13. The method according to claim 11, wherein
in the step for calculating a probability of high ovarian response, formula I for calculating the probability of high ovarian response of the subject is pre-stored for use, wherein the formula I is obtained by fitting based on data of basal anti-Mullerian hormone (basal AMH) level and change in serum inhibin B level (ΔinhibinB) of a patient receiving standard ovarian stimulation treatment in an existing database.

14. The method according to claim 12, wherein,
in the step for calculating a probability of high ovarian response, formula II for calculating the probability of high ovarian response of the subject is pre-stored for use, wherein the formula II is obtained by fitting based on data of basal anti-Mullerian hormone (basal AMH) level, change in serum inhibin B level (ΔinhibinB), basal follicle-stimulating hormone (basal FSH) level, basal androstenedione (basal AND) level and change therein (ΔAND), and basal luteinizing hormone (basal LH) level of a patient receiving the standard ovarian stimulation treatment in an existing database.

15. The method according to claim 11, wherein,
in the data collection step,
the data of the basal anti-Mullerian hormone (basalAMH) level acquired refers to a concentration of anti-Mullerian hormone in venous blood of the subject on the 2nd day of menstrual period after receiving the ovarian stimulation treatment,
the data of the change in serum inhibin B level (ΔinhibinB) acquired refers to a difference between inhibin B concentrations in the venous blood of the subject on the 6th day and the 2nd day of the menstrual period after receiving the ovarian stimulation treatment,
the data of the basal follicle-stimulating hormone (basal FSH) level acquired refers to a concentration of FSH in the venous blood of the subject on the 2nd day of the menstrual period after receiving the ovarian stimulation treatment,
the data of the basal androstenedione (basal AND) level acquired refers to a concentration of androstenedione in the venous blood of the subject on the 2nd day of the menstrual period after receiving the ovarian stimulation treatment, and the data of the change in androstenedione level (ΔAND) refers to a difference between the concentrations of androstenedione in the venous blood of the subject on the 6th day and the 2nd day of the menstrual period after receiving the ovarian stimulation treatment, or
the data of the basal luteinizing hormone (basal LH) level acquired refers to a concentration of LH in the venous blood of the subject on the 2nd day of the menstrual period after receiving the ovarian stimulation treatment.

16. The method according to claim 11, wherein,
in the step for calculating probability of high ovarian response, formula I for calculating the probability of high ovarian response probability of the subject, which is obtained by fitting based on the data of the basal anti-Mullerian hormone (basal AMH) level and the change in serum inhibin B level (ΔinhibinB) of the patient receiving the standard ovarian stimulation treatment in the existing database, is a calculation formula obtained by fitting data of the anti-Mullerian hormone (AMH) level and change in INHBB level (ΔinhibinB) of the patient receiving the standard ovarian stimulation treatment in the existing database by logistic regression,
the formula I can be used to calculate the probability of high ovarian response occurrence of the subject by using the data of the basal anti-Mullerian hormone (basal AMH) level and dynamic change in inhibin B level (ΔinhibinB) of the subject acquired by the data collection module.

17. The method according to claim 14, wherein,
in the step for calculating a probability of high ovarian response, the formula II for calculating the probability of high ovarian response of the subject, which is obtained by fitting based on the data of the the basal anti-Mullerian hormone (basal AMH) level, the change in inhibin B level (ΔinhibinB), the basal follicle-stimulating hormone (basalFSH) level, the basal androstenedione (basal AND) level and the change therein (ΔAND), and the basal luteinizing hormone (basal LH) level of the patient receiving the standard ovarian stimulation treatment in the existing database, is a calculation formula obtained by fitting the data of the basal anti-Mullerian hormone (basal AMH) level, the change in inhibin level (ΔinhibinB), the basal follicle-stimulating hormone (basal FSH) level, the basal androstenedione (basal AND) level and the change therein (ΔAND), and the basal luteinizing hormone (basal LH) level of the patient receiving the standard ovarian stimulation treatment in the existing database by logistic regression;
the formula II can be used to calculate the probability of high ovarian response of the subject by using the data of the basal anti-Mullerian hormone (basal AMH) level, the change in inhibin B level (ΔinhibinB), the basal follicle-stimulating hormone (basal FSH) level, the basal androstenedione (basal AND) level and the change therein (ΔAND), and the basal luteinizing hormone (basal LH) level of the subject acquired by the data collection module.

18. The method according to claim 16, wherein,
the formula I is:
$$P = 1 / ( 1 + {e}^{( - ( a + b * ln ( basal AMH ) + c * ln ( Δ inhibinB ) ) )} ) ,$$
wherein
p is a parameter calculated to characterize the probability of high ovarian response of the subject;
a is any value selected from a range of −18.49449 to −9.790517, preferably −14.1425;
b is any value selected from a range of 0.4227336 to 1.548814, preferably 0.9857738;
c is any value selected from a range of 0.9233156 to 2.2649428, preferably 1.5941292; and
e is a natural number.

19. The method according to claim 17, wherein,
the formula II is:
$$P = 1 / ( 1 + {e}^{( - ( d + f * basal FSH + g * ln ( basal AMH ) + {h}^{*} basal AND + i * ln ( Δ inhibinB ) + {j}^{*} Δ AND + k * basal LH ) )} ) ,$$
wherein
p is a parameter calculated to characterize the probability of high ovarian response of the subject,
d is any value selected from a range of −15.39228 to −6.729412, preferably −11.06085;
f is any value selected from a range of −0.589993 to −0.09121, preferably −0.340602;
g is any value selected from a range of 0.1606959 to 1.369763, preferably 0.7652294;
h is any value selected from a range of −0.013212 to 0.1226672, preferably 0.0547278;
i is any value selected from a range of 0.65715 to 2.0135819, preferably 1.3353659;
j is any value selected from a range of −0.036413 to 0.1330693, preferably 0.0483282;
k is any value selected from a range of −0.051137 to 0.2291053, preferably 0.088984; and
e is a natural number.

20. The method according to claim 14, wherein,
the step for calculating a probability of high ovarian response also comprises a comparison substep, in which probability values of the high ovarian response of the subject calculated by formula I and formula II respectively are compared, and a larger probability value is taken as the probability of high ovarian response of the subject.
